(19) United States
(12) Patent Application Publication
(10) Pub. No.: US 2024/0245824 A1
(43) Pub. Date: Jul. 25, 2024

(54) Shingles Remediation Belt Device

(71) Applicant: Sara Barberio, Shadow Hills, CA (US)

(72) Inventor: Sara Barberio, Shadow Hills, CA (US)

(21) Appl. No.: 18/587,467

(22) Filed: Feb. 26, 2024

(51) Int. Cl. A61L 15/42; A61L 15/26; A61L 15/44

(52) U.S. Cl. CPC A61L 15/425; A61L 15/26; A61L 15/44

(57) ABSTRACT

A shingles remediation belt device is provided which may utilize a protective layer having a outer side surface and a inner side surface opposite the outer side surface, an absorptive layer having a outer side surface and a inner side surface opposite the outer side surface, a porous barrier layer having a outer side surface and a inner side surface opposite the outer side surface, and a sanitary layer having an outer side surface and an inner side surface opposite the outer side surface. Further, the inner side surface of the protective layer may be disposed adjacent the outer side surface of the absorptive layer, the inner side surface of the absorptive layer may be disposed adjacent the outer side surface of the porous barrier layer, and the inner side surface of the porous barrier layer may be disposed adjacent the outer side surface of the sanitary layer.

### BACKGROUND OF THE INVENTION

The present invention generally relates to the field of medical devices and more particularly to the treatment and management of Herpes Zoster, commonly known as shingles, using a multilayered belt device designed to be applied around a treatment area of a user. The inventive device combines the effective delivery of medicaments specifically designed for shingles remediation with the convenience, discretion and security of a wearable belt device.

The health care and medical industry has made significant strides in improving the quality of life of individuals suffering from various diseases, including viral infections like shingles. Shingles, a reactivation of the varicella-zoster virus, is a painful rash that generally affects one side of the body. Despite the availability of vaccines to prevent the occurrence of shingles, there are a substantial number of people who still experience the disease due to factors such as age, weakened immune system, or those who were unable to get vaccinated.

Treatment of shingles primarily revolves around antiviral medications and pain management. Conventionally, antiviral medications are taken orally, and topical ointments, creams, or patches are used for localized relief. However, these existing products have several inadequacies. The oral administration of antiviral medications often leads to systemic side effects. Topical applications, on the other hand, are difficult to self-administer especially in hard-to-reach areas of the body, and they can be easily rubbed off by clothing or sheets, reducing their effectiveness over time. Furthermore, patches can only cover a limited area and may not be able to conform to the contours of the body, resulting in uneven distribution of the medication and incomplete relief.

Notably, a substantial limitation of existing products in the market pertains to the inability to accommodate the irregular shapes and patterns in which shingles outbreaks occur. Shingles rashes typically form a band around one side of the body or face, but the shape and extent can significantly vary among patients. Therefore, a ‘one size fits all’ approach, such as that offered by conventional patches, is often inadequate. Therefore, there is a need in the market for a belt device that can accommodate irregularly-shaped rashes of varying size.

Existing topical products are typically designed for relatively uniform, flat surfaces, failing to account for the unique, individualized morphology of shingles outbreaks. This lack of adaptability often results in insufficient coverage of the rash, hindering effective medication delivery and healing process. Moreover, existing products do not offer any modularity, that is, the capability of coupling multiple belts or patches together to accommodate larger or irregularly shaped affected areas. This lack of customization and flexibility, compounded by the awkwardness and discomfort experienced by the patients during application, results in suboptimal patient adherence to the treatment regimen.

The current state of the art does not provide a solution that can easily be adapted to the patient's specific needs, which underscores the necessity for a more personalized, adaptable, and efficient method of treatment. There exists a need for a shingles treatment device that addresses the limitations of the current methods. A more customizable-shaped, localized, consistent, and effective delivery of shingles remediation agents is required to minimize the side effects and maximize the therapeutic benefit.

The proposed belt device can be comfortably worn around the body, covering a larger area than traditional patches, ensuring that the affected region receives a steady and controlled release of the medicament. The device adheres securely to the skin, minimizing the chance of the medicament being rubbed off and maximizing therapeutic exposure. Furthermore, the belt-like design allows the user to independently apply the treatment, even to hard-to-reach areas and irregularly-patterned rashes, making it an empowering tool for those suffering from shingles. The innovative combination of gauze, porous bandage layer, and shingles remediation agents also offers potential for enhanced comfort and healing when compared to existing solutions. In view of the above, it can be seen that the present invention provides a significant improvement over existing products in terms of convenience, effectiveness, applicability, and patient empowerment.

### BRIEF DESCRIPTION OF THE DRAWINGS

FIG. 1 illustrates a plan view of a top surface of a shingles remediation belt device in a patch configuration in accordance with some embodiments of the present invention.

FIG. 2 illustrates an exploded perspective view of a multilayered stack of a shingles remediation belt device in accordance with some embodiments of the present invention.

FIG. 3 illustrates a detailed view of a gauze layer of a shingles remediation belt device in accordance with some embodiments of the present invention.

FIG. 4 illustrates a plan view of a modularly coupled configuration of a plurality of shingles remediation belt devices in accordance with some embodiments of the present invention.

FIG. 5 illustrates an overview of a protective layer being removed from a bottom surface of a shingles remediation belt device in accordance with some embodiments of the present invention.

FIG. 6A illustrates a plan view of a top surface of an elongated configuration of a shingles remediation belt device in accordance with some embodiments of the present invention.

FIG. 6B illustrates a partial overview of two ends of an elongated configuration of a shingles remediation belt device secured together in an overlapping coupling configuration in accordance with some embodiments of the present invention.

FIG. 7 illustrates a cross-sectional view of a multilayer stack of a shingles remediation belt device enclosed within a product packaging envelope in accordance with some embodiments of the present invention.

### DETAILED DESCRIPTION OF THE INVENTION

Before describing the present invention in detail, it is to be understood that the invention is not limited to any one of the particular embodiments, which of course may vary. It is also to be understood that the terminology used herein is for the purpose of describing particular embodiments only, and therefore is not necessarily intended to be limiting. As used in this specification and the appended claims, terms in the singular and the singular forms “a,” “an,” and “the” include plural referents unless the context clearly dictates otherwise. Thus, for example, reference to “a shingles remediation belt device” also includes a plurality of shingles remediation belt device and the like.

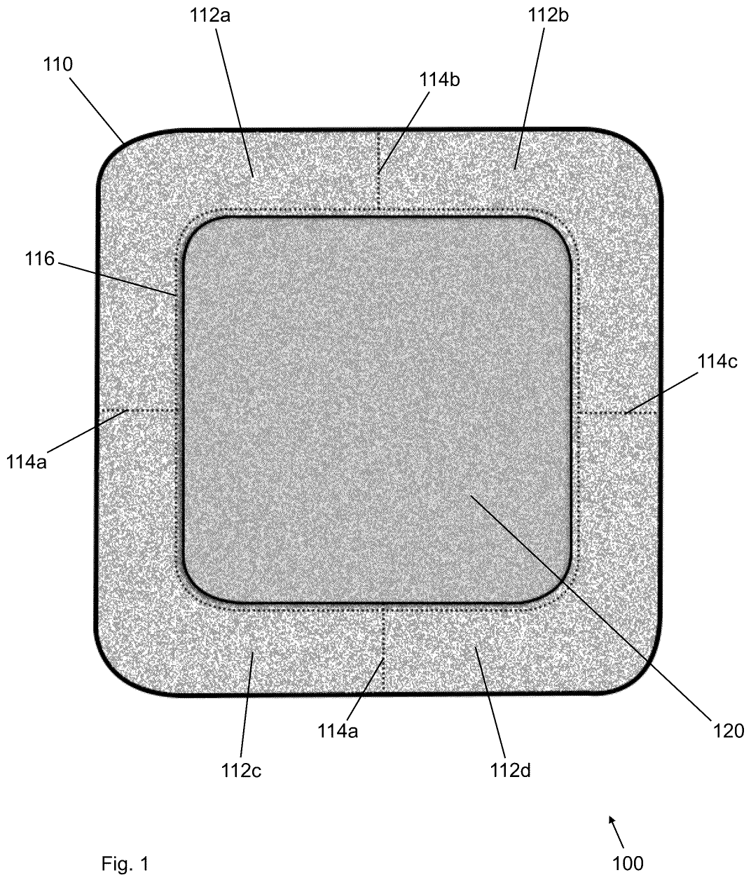

Exemplary embodiments of the present invention are illustrated in the accompanying figures. As shown in FIG. 1, a plan view of a top surface of a shingles remediation belt device 100 in a patch configuration is provided. The patch configuration of the shingles remediation belt device 100 may comprise a perimeter section 110 and an interior section 120 disposed within the perimeter section 110. The perimeter section 110 may comprise a plurality of coupling portions 112a-112d that may cover the entirety of the perimeter section 110 in a successively arranged adjacent manner as shown in FIG. 1.

Each of the plurality of coupling portions 112a-112d may comprise a first multilayered structure, where each layer of a given coupling portion 112a-112d is, in part, designed to facilitate remediation of a shingles rash outbreak. Further, the plurality of coupling portions 112a-112d may be non-permanently adjoined to a successive adjacent coupling portion such that coupling portion 112a may be non-permanently adjoined to coupling portions 112b, 112c and so on as shown in FIG. 1. The non-permanent connection between the plurality of coupling portions 112a-112d may be achieved via respective removal elements 114a-114d which are designed to facilitate the removal of one or more layers from the first multilayered structures of the plurality of coupling portions 112a-112d. Further, a perimeter removal element 116 may be disposed along the perimeter of the interior section 120 and orthogonal to each of the removal elements 114a-114d.

Each of the removal elements may be fabricated as perforation or an incision into and/or through an outer layer of the first multilayered structures. Thereby, the removal elements may be removed easily by a user in order to expose an adhesive layer of the first multilayered structure of each coupling portion, where the adhesive layer is illustrated in FIG. 1 as the textured interior of each of the coupling portions 112a-112d. This allows the adhesive layer of the coupling portions 112a-112d to be to be adhered to a user's skin around an area containing a shingles rash outbreak. Specifically, the interior section 120 may be exposed to and in direct adjacent contact with the area containing the singles rash outbreak.

The interior section 120 may comprise a plurality of layers fabricated into a second multilayered structure which is distinct from the first multilayered structures, where the first multilayered structures of each of the coupling portions 112a-112d may be structurally monolithic and continuous with one another. The second multilayered structure may comprise one or more layers which are structurally monolithic and continuous with a layer of the first multilayered structures. Further, the second multilayered structure of the interior section 120 may comprise one or more layers that does not exist in the first multilayered structure of the coupling portions 112a-112d of the perimeter section 110, and vice versa.

In some embodiments, the first multilayered structures may comprise at least one of a protective layer, an adhesive layer, a sanitary layer and the like or any combination thereof. One or more layers of the first multilayered structures of the plurality of coupling portions 112a-112d of the perimeter section 110 may not extend into the interior section 120. The second multilayered structure may comprise at least one of a protective layer, an absorptive layer, a porous barrier layer, a sanitary layer and the like or any combination thereof. One or more layers of the second multilayered structure of the interior section 120 may not extend into any of the plurality of coupling portions 112a-112d of the perimeter section 110.

In other embodiments, the first multilayered structures and the second multilayered structure can include a therapeutic agent layer. This therapeutic agent layer could contain shingles remediation agents or medications that are effective against the symptoms of shingles, such as antiviral drugs, anesthetics, or anti-inflammatory drugs. This layer may directly contact the affected area when the device is applied to the user's skin. The agents could be incorporated into a biocompatible matrix that permits sustained, controlled release of the agents over an extended period, enhancing the therapeutic effect and providing relief from symptoms.

Moreover, entirety of the shingles remediation belt device 100 could have a modular design. This modularity could allow the user to adjust the size of the belt device 100 by adding or removing sections according to the extent and irregular shape of the shingles rash. This modular design addresses one of the key limitations of conventional patches, providing superior customization to the user's specific needs. The respective removal elements 114a-114d could facilitate this modularity, allowing the user to easily detach or add a section of the device 100 when required.

Specifically, if the removal elements 114a-114d are fabricated as perforations and the perforations are disposed through each of the layers of the first and second multilayered structures of the perimeter and interior sections 110, 120, then the device 100 may be easily torn by the user into fractional sections such as halves or quarters in order to allow the user to adjoin respective edges of second multilayered structures of a first whole device 100 and a second fractionally-sectioned device 100 which expands the shape of the medically-active area of the second multilayered structure of the first whole device 100 which is relied upon to treat a shingles rash outbreak when placed adjacent the entirety of the rash outbreak areas. In such a configuration, at least a part of one or more of the coupling portions 112a-112d of the first whole device 100 may be disposed over the second multilayered structure of the second fractionally-sectioned device 100.

The first multilayered structures could also feature a layer of biocompatible, skin-friendly adhesive within the adhesive layer that not only ensures secure attachment of the device 100 to the skin but also minimizes the risk of skin irritation. Additionally, a moisture-absorbing material layer could be integrated into the first multilayered structures to maintain the bond between the adhesive layer and the user's skin which promotes healing and provides additional comfort and confidence to the user that the device 100 will not fall off the user upon getting wet or sweating.

The adhesive layer of the shingles remediation belt device 100 can be implemented using a skin-mimicking tape which is typically used in facial kinesiology but unobviously providing several unique benefits to the context of the shingles remediation belt device 100. This skin-mimicking tape adhesive layer may be particularly suited to applications on the skin due to its texture, ease of removal, and strong adhesive properties. It is made from a blend of high-quality, medical-grade adhesives, ensuring it is appropriate for use even on sensitive or acne-prone skin.

Further, this skin-mimicking tape adhesive layer, while being typically used for facial applications, may be equally suited for application to the various contours of the user's body and torso while also accommodating bodily movements without risking adhesive failure of the remediation belt device 100. The breathability of this tape is superior due to its composition of between 85% and 95% organic cotton and between 5% and 15% elastane, mimicking the texture and elasticity of human skin. This quality ensures that the belt device 100 remains comfortable and breathable when wrapped around the user's body, reducing potential irritation and discomfort.

The skin-mimicking tape adhesive layer's hypoallergenic and latex-free properties make it appropriate for all skin types, including sensitive skin, making the remediation belt device 100 a versatile tool for a broad population of users. This adhesive layer is designed to be easily removed, minimizing discomfort during detachment. It is also important to note that the adhesive layer is applied without stretching it to the pre-extended area of skin or muscle parts. This characteristic enables a secure attachment of the belt device 100 while still allowing for the flexibility and movement of the user.

The shingles remediation belt device 100 may comprise an adhesive layer infused with one or more medicinal agents to augment its therapeutic efficacy. These medicinal agents could encompass anti-inflammatory compounds, analgesics, antiviral substances, or other therapeutics suitable for treating shingles. Incorporation of the medicinal agents into the adhesive layer could be achieved by a variety of techniques, including direct impregnation or solvent casting. In the latter method, the medicinal agent is mixed with a solvent, and the solvent is subsequently evaporated, leaving the medicinal agent embedded within the adhesive layer. This infusion process is tailored to permit a controlled and steady release of the medicinal agents over an extended period. The sustained release functionality ensures a continuous delivery of therapeutic agents to the affected skin area throughout the period of application, contributing to an efficient healing process.

From a transdermal absorption standpoint, the infused medicinal agents can migrate from the adhesive layer to the skin due to a concentration gradient. The skin acts as a semi-permeable membrane, allowing the medicinal agents to penetrate the skin layers and reach the affected site. The degree and rate of this transdermal absorption depend on several factors, including the physical and chemical properties of the medicinal agents, the condition of the skin, and the characteristics of the adhesive layer. The inclusion of medicinal agents directly in the adhesive layer enhances the versatility and efficacy of the shingles remediation belt device, offering not only physical support but also ongoing, targeted medicinal treatment.

In other embodiments, a smart layer could be integrated into either the first multilayered structures or the second multilayered structure. This smart layer could contain sensors or chemically active visual indicators to monitor parameters such as temperature, humidity, or pH that may affect the healing process. The visual indicator may enable the user or healthcare provider to monitor the sanitary status of the belt device 100. This may allow the user or healthcare provider to know when to replace the existing belt device 100 with a new belt device 100 to continue optimal treatment of the shingles rash outbreak.

In various embodiments of the invention, a multitude of natural and beneficial medicinal agents could be absorbed, impregnated, or applied into the porous barrier layer (in some embodiments is fabricated as a biocompatible polymer matrix layer) and/or the absorptive layer (in some embodiments is fabricated as at least one layer of gauze). Predominantly, the primary natural topical healing agents incorporated within these layers could include aloe vera, Manuka honey, and coconut oil. These are known for their natural healing properties, providing relief from the discomfort and pain associated with shingles, as well as promoting skin healing.

The porous barrier layer and the absorptive layer may also contain additional beneficial agents that could aid in the healing and relief process. Such agents could include coco butter and shea butter, which moisturize skin and provide anti-inflammatory properties; olive oil and hemp oil, which can help to nourish and soothe the skin; and oat derivatives and plumeria oil, which may have soothing and calming effects on irritated skin.

Further, the invention could incorporate other beneficial natural agents within the layers. These could include melatonin, which may provide antioxidant and anti-inflammatory properties; St. John's Wort, a natural remedy that may ease skin inflammation; oregano oil, which may provide antimicrobial properties; and Echinacea, lemon balm, and green tea, which may provide immune-boosting properties. Essential fatty acids could be included for their role in maintaining skin health, and tea tree oil and garlic for their antiviral properties. Additionally, vitamins such as Vitamin C and Vitamin D could be included for their roles in skin health and immune function. Oatmeal, a soothing agent, could also be incorporated. All of these agents could be used individually or in combinations to achieve optimal therapeutic effect.

The medicinal agents may be selected and combined in such a manner as to optimize the healing and symptomatic relief process for individuals suffering from shingles. The presence of these agents within the porous barrier layer and the absorptive layer ensures that they are directly applied to the affected area, maximizing their therapeutic effect and enhancing the overall efficacy of the shingles remediation belt device 100.

In the context of the porous barrier layer, the term ‘network structure’ may be utilized to broadly embody various configurations including, but not limited to, a matrix structure and a mesh structure. A network structure, as the name suggests, entails an interlinked assembly of nodes and links. In the case of a matrix, the network structure might be thought of as a regular, tightly packed arrangement of constituents, potentially filled with medicinal agents. This densely structured form provides a higher surface area for interaction with the healing wound, which can be beneficial for the delivery of medicinal agents and the absorption of exudates.

On the other hand, a mesh configuration may be implemented as an open-weave, more loosely constructed network, akin to a net or lattice. The open voids within the mesh structure allow for superior airflow, promoting a healthy and well-ventilated healing environment. Moreover, the mesh can accommodate larger particulates and allow for more significant fluid flow, which can be advantageous in managing heavily exudating wounds. Thus, a network structure, manifesting as either a matrix or a mesh, adds a degree of versatility to the porous barrier layer, enabling it to cater to various wound healing scenarios.

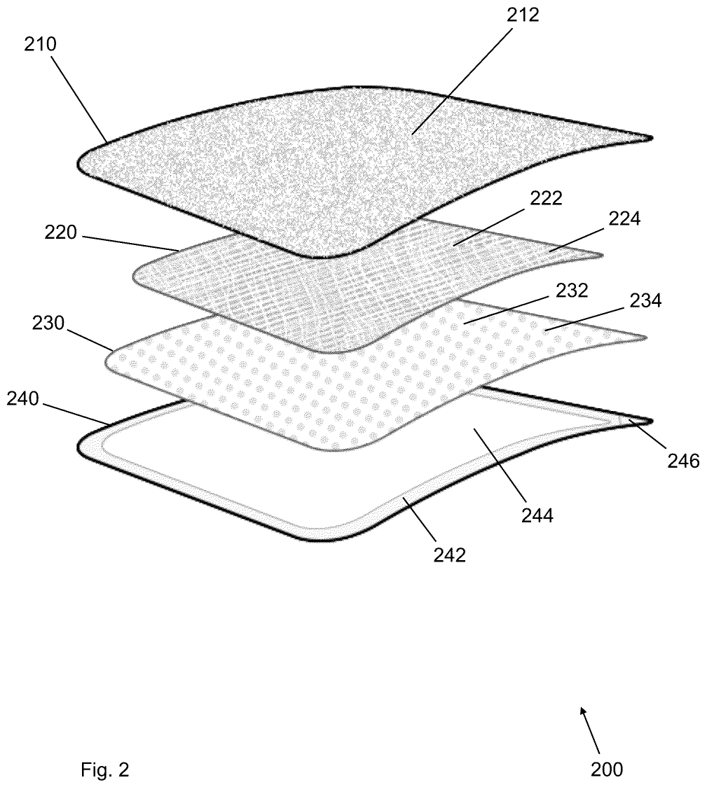

As shown in FIG. 2, an exploded perspective view of a multilayered stack of a shingles remediation belt device 200 is provided. The shingles remediation belt device 200 may comprise a protective layer 210, an absorptive layer 220, a porous barrier layer 230 and a sanitary layer 240. In some embodiments, each of the layers 210, 220, 230, 240 may comprise a planar shape with a plurality of straight edges. Further, the protective layer 210 may be exposed adjacent the external environment on a first side or may be disposed adjacent another sanitary layer 240 disposed thereover prior to its removal. A second side of the protective layer 210 may be disposed adjacent a first side of the absorptive layer 220. The second side of the absorptive layer 220 may be disposed adjacent a first side of the porous barrier layer 230. The second side of the porous barrier layer 230 may be disposed adjacent a first side of the sanitary layer 240. Before removal, a second side of the sanitary layer 240 may be disposed adjacent the external environment. After removal, the second side of the porous barrier layer 230 may be disposed adjacent a user skin area afflicted with a shingles rash outbreak.

The protective layer 210 may comprise a material body 212 that is fabricated from a low-friction flexible material in order to be robust against the wear and tear of bodily use. For instance, it is advantageous that the material body 212 not get caught or snagged or abraded against an incident object or surface while being worn by the user. If this were to happen, then the user would be subject to the painful experience of their healing shingles rash outbreak area being agitated and disrupted during the healing process, thereby causing a delay in the healing process and a resetting of any scabs that were in the process of forming.

The absorptive layer 220 may comprise general mesh structure that comprises a fabric body 222 having a plurality of apertures 224 formed therein. Such a mesh structure allows any bodily fluids emitted by the healing shingles rash outbreak to pass through the porous barrier layer 230 and then to be absorbed by the absorptive layer 220 while still being exposed to evaporative and oxidative environmental air via the breathable protective layer 210. Accordingly, it is important that a matrix 232 having pores 234 of the porous barrier layer 230 comprise a material that does not allow it stick to the afflicted area of the user's skin having the shingles rash outbreak so as to allow the device 200 to be removed easily without disturbing the scab formation and healing processes.

While only one sanitary layer 240 is illustrated, it is understood that another sanitary layer may be utilized above the protective layer 210 such that each of the sanitary layers 240 may be non-permanently coupled together along a perimeter adhesive area 242 which surrounds a material interior area 244 that covers the entirety of the layers 210, 220, 230 as shown in FIG. 2. The removal tab 246 may be employed along one of the corners of the sanitary layer 240 in order to facilitate the removal of the one or more sanitary layers 240 which thereby puts the device 200 in an exposed application configuration from its original enclosed sanitary configuration prior to removal of the one or more sanitary layers 240.

Extending the features of the protective layer 210 further, it becomes crucial to consider the inherent dynamism of the human body and the corresponding adaptability of the material body 212. The human body, specifically in the torso region where the device 200 might often be applied, exhibits a cyclic pattern of expansion and contraction due to the natural process of breathing. As such, the material body 212 needs to be designed with the capability to mirror this movement without interrupting the healing process of a shingles rash outbreak.

In light of this, the material body 212 is conceived to exhibit an impressive degree of flexibility, capable of expanding and contracting in harmony with the user's breathing cycle. Normal breathing results in an average chest wall surface area expansion of approximately 35% to 40% in healthy adults. It is therefore beneficial for the material body 212 to be capable of expanding by at least this degree. This functional adaptation allows the shingles remediation belt device 200 to remain continuously in place and in contact with the rash area without applying excess pressure or causing dislocation.

Moreover, such flexibility prevents any interruption in the scab-forming process, which is a crucial step in the healing of a shingles rash outbreak. Therefore, the low-friction flexible material of the material body 212 not only serves to protect against wear and tear but also plays a significant role in ensuring consistent and non-interruptive contact with the rash outbreak area. This, in turn, allows for the uninterrupted remediation process, thus contributing to the overall effectiveness of the shingles remediation belt device 200.

Similarly, the absorptive layer 220 and the porous barrier layer 230 should ideally possess the same degree of flexibility and stretchability as the protective layer 210. The need for such physical adaptability becomes essential when considering the physiological dynamics associated with the human body during a normal breathing cycle. As the surface area of the skin expands and contracts, it is crucial that these layers retain their positional stability over the afflicted area while simultaneously allowing for the necessary degree of deformation. Therefore, an optimal absorptive layer 220 and porous barrier layer 230 should also have an expansion capability of around 35% to 40% to accommodate the body's natural movements, without causing any disturbance to the healing process. This flexibility ensures that the layers remain in constant contact with the skin, thereby maintaining the delivery of the medicinal agents to the shingles outbreak area and facilitating uninterrupted wound healing. It also ensures that these layers maintain their integrity and do not tear or rupture during use, thereby ensuring the consistent performance of the shingles remediation belt device 200.

In its sanitary enclosed configuration, the shingles remediation belt device 200 presents a layered stack comprising a protective layer 210, an absorptive layer 220, a porous barrier layer 230, and a sanitary layer 240. In this configuration, each of these layers maintains a planar shape with multiple straight edges, contributing to the modular and adaptable form factor of the device. The sanitary layer 240 is positioned such that one side is adjacent to the porous barrier layer 230 and the other side faces the external environment. This external-facing sanitary layer 240 serves to keep the underlying layers sterile prior to the application of the device. Additionally, the protective layer 210 may also have an associated sanitary layer disposed over its exposed first side, serving as an additional protective measure for the underlying layers.

In the exposed application configuration, the sanitary layer(s) 240 are removed, which allows for the direct contact of the porous barrier layer 230 and, optionally, the protective layer 210 with the user's skin. Upon removal of the sanitary layer 240, the second side of the porous barrier layer 230 can be applied directly to the user's skin area afflicted with a shingles rash outbreak. This enables the medicinal agents absorbed or impregnated into the absorptive layer 220 and porous barrier layer 230 to have direct contact with the affected skin, facilitating their therapeutic action. Similarly, if an additional sanitary layer was present over the protective layer 210, its removal would allow the protective layer to serve its purpose in providing a shield against external influences. In this configuration, the shingles remediation belt device 200 is primed for therapeutic effectiveness, keeping the affected area sterile, protected, and in direct contact with the beneficial medicinal agents.

Generally, the protective layer 210 may be fabricated from a low-friction flexible breathable non-adsorptive and at least partially-hydrophobic material. In certain embodiments of the invention, the protective layer 210 can be fabricated from a variety of materials, each possessing unique properties that lend themselves to advantageous use in the device 200. Such materials include, but are not limited to, polypropylene, polyethylene, polyurethane, polyester, PTFE (Polytetrafluoroethylene), silicone-coated fabrics, neoprene, TPE (thermoplastic elastomer) films and the like or any combinations thereof.

Polypropylene is a synthetic polymer that may be used due to its hydrophobicity, breathability, and low friction. This material is also known for its lightweight and flexible nature, which may contribute to the comfort and efficacy of the shingles remediation belt device 200. Polyethylene, another synthetic polymer, may also serve as a suitable material for the protective layer 210, given its notable flexibility, water resistance, and low friction properties. Polyurethane may be utilized in various forms, such as films and foams, and can be treated to enhance its hydrophobicity, thereby providing the desired flexibility and breathability for the protective layer 210. In a similar vein, polyester, especially when coated or treated, may also meet the requirements for breathability, flexibility, and water resistance. PTFE (Polytetrafluoroethylene), with its characteristic low friction and hydrophobic properties, may serve as another potential material for the protective layer 210. Additionally, silicone-coated fabrics may offer flexibility, breathability, and water resistance, aligning with the desired properties for this layer. Neoprene, a type of synthetic rubber, due to its flexibility and water resistance, may also be used for the protective layer 210, bearing in mind the breathability requirements of the device. TPE films are a type of plastic that have rubber-like properties. TPE films are flexible, durable, and lightweight, and can be made in a variety of textures

It should be understood that the aforementioned materials are examples, and the selection for the protective layer 210 may be based on specific requirements of the device, taking into consideration factors such as cost, availability, ease of fabrication, and user comfort. Other materials possessing the requisite properties of low friction, flexibility, breathability, non-adsorptiveness, and at least partial hydrophobicity could also be employed without deviating from the spirit of the invention.

In certain embodiments, the shingles remediation belt device 200 may have two sanitary layers 240, one each enveloping the top and bottom surfaces of the device, forming a comprehensive preservative seal around the layered structure. The dual sanitary layers 240 serve to encase the entirety of the device, isolating the inner layers and their medicinal agents from the external environment. This sealed configuration ensures that the medicinal agents within the absorptive layer 220 and the porous barrier layer 230 are well-preserved and protected from potential degradative environmental factors such as air, light, and moisture. By preventing premature exposure of the medicinal agents, the sanitary layers 240 help maintain the potency, efficacy, and shelf-life of these agents. Upon removal of the sanitary layers 240, the device 200 is ready for application, with the medicinal agents kept at their optimum therapeutic value, enhancing the overall effectiveness of the shingles remediation belt device in providing relief to individuals suffering from shingles.

The absorptive layer 220, integral to the shingles remediation belt device 200, may be composed of a variety of sanitary absorbent materials. These materials must ensure a high level of hygiene while also serving to absorb any discharge resulting from a shingles outbreak. This layer could be fabricated as a single sheet of material that extends across the length of the protective layer 210, concentrated specifically along the central portion of the protective layer's second side.

Cotton, a highly absorbent natural fiber, can serve as an ideal candidate for the absorptive layer 220, given its softness, comfort, and impressive moisture-wicking abilities. It may provide an adequate barrier between the shingles rash and the other layers of the device, absorbing and containing any excess fluid. Rayon, a semi-synthetic material, could be another suitable choice for the absorptive layer 220. Recognized for its high absorbency, soft texture, and versatility, rayon may provide comparable performance to cotton while potentially offering a more cost-effective solution. Polyester, another synthetic fiber, might be employed due to its durability and versatility. When combined with other fibers in a blend, polyester can enhance the durability and absorbency of the material, thus increasing the efficacy of the device.

Beyond these examples, other materials may also be employed without deviating from the spirit of the invention, such as linen, bamboo fibers, or cellulose-based materials. Linen, a natural fiber known for its exceptional absorbency and durability, could provide an excellent balance of performance and comfort. Bamboo fibers, recognized for their natural absorbency, softness, and antimicrobial properties, may offer additional benefits, especially for sensitive skin. Cellulose-based materials, being highly absorbent and biodegradable, could provide an eco-friendly alternative.

It should be noted that these material choices are non-limiting examples, and the selection for the absorptive layer 220 can be tailored according to the specific requirements of the device, including but not limited to factors such as cost, availability, ease of fabrication, absorbency, and user comfort. The spirit of the invention encompasses the use of any material that can provide effective absorption while maintaining an adequate level of hygiene and comfort for the user.

The porous barrier layer 230 forms an integral part of the shingles remediation belt device 200. It is designed to be disposed adjacent to a user's skin area afflicted with a shingles rash outbreak. This layer can be fabricated from a multitude of material options, each offering a unique set of properties that can contribute to the effectiveness of the device. Silicone-based materials can serve as a preferred choice for the porous barrier layer 230, due to their high biocompatibility, flexibility, and moisture-resistant properties.

Specifically, a silicone-based mesh-like material could serve to create an interface that is gentle on the skin while allowing optimal permeability for the medicinal agents.

Polyamide, a synthetic resin, can provide an alternative due to its strength, flexibility, and resistance to wear and tear. It can be processed into a nonwoven fabric, offering breathability and moisture transfer capabilities. Moreover, a silicone-based adhesive could be integrated into this fabric, creating a gentle, non-stick surface that protects the afflicted area. Cellulose acetate, a biodegradable and breathable material, could also be utilized in creating a knitted fabric for the porous barrier layer 230. This material can be impregnated with a petrolatum emulsion, which provides a non-stick surface over the wound and contributes to the barrier layer's non-adhering qualities.

Furthermore, other synthetic materials such as acrylic, nylon, polyester, and Teflon, known for their strength, durability, and flexibility, may be selected. These materials can be crafted into a mesh-like structure, providing the required permeability for the application of medicinal agents. Hydrocolloids, a group of gel-forming agents, can serve as additional options, given their ability to maintain a moist wound environment that promotes healing. Other natural fibers such as cotton or rayon could also be considered due to their high absorbency, breathability, and biocompatibility.

It should be noted that the material selection for the porous barrier layer 230 is not limited to the options listed. The ideal choice will depend on various factors, including cost, availability, ease of fabrication, compatibility with the medicinal agents, and user comfort. The spirit of the invention allows for the use of any material that aligns with the device's functional requirements while offering a high level of biocompatibility and skin-friendly properties.

The sanitary layers 240 form a key component of the shingles remediation belt device 200, providing a protective barrier that safeguards the other layers from contamination prior to use. These layers can be fabricated from a wide range of materials, each selected based on their ability to prevent the ingress of contaminants and maintain the sterility of the device 200.

Polyethylene films, being lightweight, resistant to moisture, and impermeable to bacteria, serve as a viable choice for the sanitary layers 240. Polyethylene, as a thermoplastic, also offers the advantage of being moldable into custom shapes, making it suitable for the varied dimensions of the shingles remediation belt device 200. Another suitable material is silicone-coated release paper due to its ability to provide a sterile surface. Its non-stick properties and biocompatibility make it an ideal choice for the sanitary layer. Silicone-coated release paper can prevent adhesive components from prematurely sticking and can maintain the stability of the medicinal agents, ensuring their efficacy until application. In addition to these materials, other plastic-based films like polypropylene or polyvinyl chloride (PVC) can also be utilized. Both offer high resistance to chemicals, puncture and tear resistance, and excellent barrier properties against microbes, dust, and other contaminants.

The selection of the material for the sanitary layers 240 ultimately depends on the desired properties, such as sterility, barrier performance, cost-effectiveness, and ease of removal. In all instances, the material should be compatible with the other components of the shingles remediation belt device 200, and ensure the preservation of the medicinal agents incorporated within the device 200.

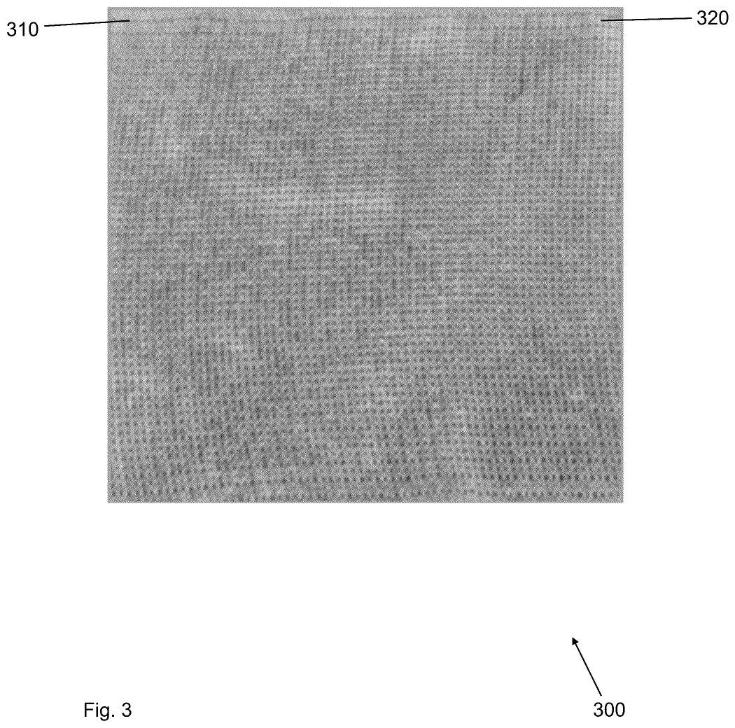

As shown in FIG. 3, a detailed view of a porous barrier layer 300 of a shingles remediation belt device is provided. The porous barrier layer 300 may comprise a plurality of pores 310 defined by a matrix 320. The pores 310 and matrix 320 of the porous barrier layer 300 may comprise a plurality of structural and materials parameters associated therewith. Specifically, the porous barrier layer 300 may exhibit specific properties related to pore diameter, pore shape, pore distribution, porosity, interconnectivity, tensile strength, elongation at break, tear resistance, puncture resistance, Young's modulus, biocompatibility and durability.

In terms of structural parameters, the porous barrier layer 300 comprises numerous pores 310 with diameters that could suitably range from 0.1 micrometers (μm) to 200 μm, with a more preferred diameter range of 1 μm to 100 μm. This narrowed range still enables efficient transmission of medicinal agents, while preserving the strength and integrity of the matrix 320. While pore shapes may vary, maintaining a predominantly circular or elliptical form may be beneficial for the uniformity in substance distribution. Specifically, shapes which exhibit a higher degree of roundness are less likely to detrimentally impact the mechanical strength of the matrix 320. Uniform pore distribution across the matrix 320 is an ideal attribute, supporting a homogenous dispersion of the healing agents across the skin and facilitating even treatment of the affected area. The porosity of the matrix 320, or the volume fraction occupied by the pores 310, may favorably lie between 20% and 80%, with a more preferred range being between 30% and 70%. This preferred range guarantees that the porous barrier layer 300 retains its structure while providing sufficient space for the transportation of the medicinal agents. The interconnectivity of pores 310 might be a key structural feature for the porous barrier layer 300, with a higher degree of interconnectivity enabling smoother flow and even distribution of medicinal agents across the layer.

Looking at the material parameters of the porous barrier layer 300, its tensile strength could beneficially range from 1 MPa to 100 MPa, with a preferred range of 10 MPa to 70 MPa. This more narrowed range ensures the layer can effectively withstand external forces without undergoing a tear or rupture. The elongation at break for the matrix material might advantageously range from 100% to 800%, with a preferred range of 200% to 600%. This denotes the material's flexibility and its resilience to stretching without breaking. The tear resistance of the matrix 320 is a crucial attribute in preventing accidental damage during application or removal. A suitable range may be between 500 N/mm and 4000 N/mm, with a preferred range between 1000 N/mm and 3000 N/mm, providing added durability to the layer. Puncture resistance is another critical material parameter for the porous barrier layer 300. An advantageous range may lie between 10 N to 200 N, with a preferred range between 20 N to 150 N, to prevent accidental penetration and ensure the integrity of the layer. The Young's Modulus for the matrix 320 material, indicating its stiffness, could ideally range from 0.1 MPa to 2 MPa, with a preferred range of 0.2 MPa to 1 MPa. This preferred range establishes a balance between flexibility and firmness. Regarding biocompatibility, the materials used for the porous barrier layer 300 should comply with ISO 10993 or similar standards, minimizing the chance of provoking an immune response or causing skin irritation. Lastly, the durability of the porous barrier layer 300 should be sufficient to endure the intended period of use, depending on the severity of the shingles outbreak and the frequency of dressing changes. Suitable materials for the matrix 320 should withstand wear, resist degradation, and perform consistently across a variety of environmental factors experienced by the layer 300.

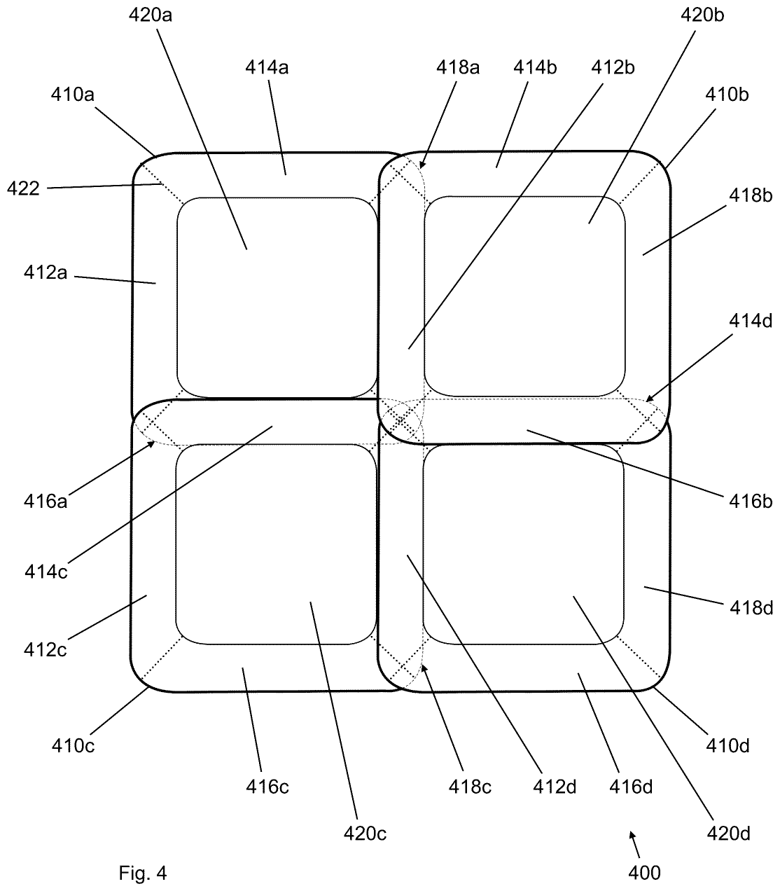

As shown in FIG. 4, a plan view of a modularly coupled configuration 400 of a plurality of shingles remediation belt devices 410a-410d is provided. The plurality of shingles remediation belt devices 410a-410d may each comprise a left coupling portion 412a -412d, a top coupling portion 414a-414d, a bottom coupling portion 416a-416d and a right coupling portion 418a-418d which are each separated by a plurality of removal elements 422. Further, each belt device 410a-410d may comprise an interior section 420a-420d surrounded by the various aforementioned coupling portions around the perimeter thereof as illustrated in FIG. 4.

The modularly coupled configuration 400 may be defined by at least one of the various coupling portions being overlapped upon another of the coupling portions. Specifically, FIG. 4 illustrates the bottom and right coupling portions 416a, 418a being overlapped by the top coupling portion 414c and left coupling portion 412b, respectively, the right coupling portion 418c overlapped by the left coupling portion 412d, and the top coupling portion 414d overlapped by the bottom coupling portion 416b. Each of the coupling portions may comprise a multilayered structure having a protective layer, an adhesive layer, a buffer removal layer, an absorptive layer, a porous barrier layer and the like or any combination thereof.

In order to achieve the modularly coupled configuration 400, any coupling portion that overlaps another coupling portion should have each of the buffer removal layer, the absorptive layer and the porous barrier layer removed in order to expose the adhesive layer for adhesion to the protective layer of the coupling portion that it is overlapping. However, the coupling portion that is being overlapped may not have any of its layers removed so that the porous barrier layer is directly in contact with the user's skin are afflicted with a shingles rash outbreak. In such a configuration, the multilayered structure of the coupling portion being overlapped may be disposed adjacent the multilayered structure of the interior section of the adjacent belt device that comprises the coupling portion (e.g. 414c) that overlaps the coupling portion (e.g. 416a) being overlapped, thereby creating a continuous multilayered structure across the user's skin surface in order to effectively remediate a shingles rash outbreak in whatever shape or pattern it presents.

As an enhancement to the modularly coupled configuration 400, the design can incorporate the feature of customization to align with the irregular shape of any shingles rash outbreak. The removability of the belt devices 410a-410d enables an adjustable configuration that conforms to the contours of the rash-affected area. The modularity of this configuration ensures a comprehensive treatment area coverage, which could otherwise be limited by the static designs of conventional wound dressings.

Furthermore, the modularly coupled configuration 400 offers a unique flexibility, allowing for reconfiguration of the coupled belts 410a-410d in response to the evolving nature of the shingles rash outbreak. If the rash spreads or shrinks, the belt devices can be added or removed as required to match the new rash pattern, effectively maintaining full coverage without wastage or insufficient treatment area.

The removal elements 422, which may take the form of perforations, scoring, or other easily severable points incorporated into the structure of the belt devices 410a-410d, provide another level of flexibility and adaptability for the user. These elements allow for the customization of the shape and size of each belt device, enabling the user to tear or cut away sections of the device to accommodate the unique, irregular shape of a shingles rash outbreak.

By leveraging these removal elements 422, users can create a variety of configurations from a single belt device, including but not limited to fractional segments such as halves, quarters, or even smaller sections as needed. This could be particularly advantageous for adapting to a rash outbreak that is not perfectly covered by the standard full size of the belt device, ensuring that all affected areas are adequately addressed.

Moreover, this ability to adjust the shape and size of the belt devices via the removal elements 422 extends to the modularly coupled configuration 400 as well. By using fractional portions of different belt devices, a user can create a composite layout that closely mirrors the area and shape of the shingles rash outbreak. Such an approach further enhances the effectiveness of the shingles remediation belt device, promoting a more efficient and comfortable healing process for the user.

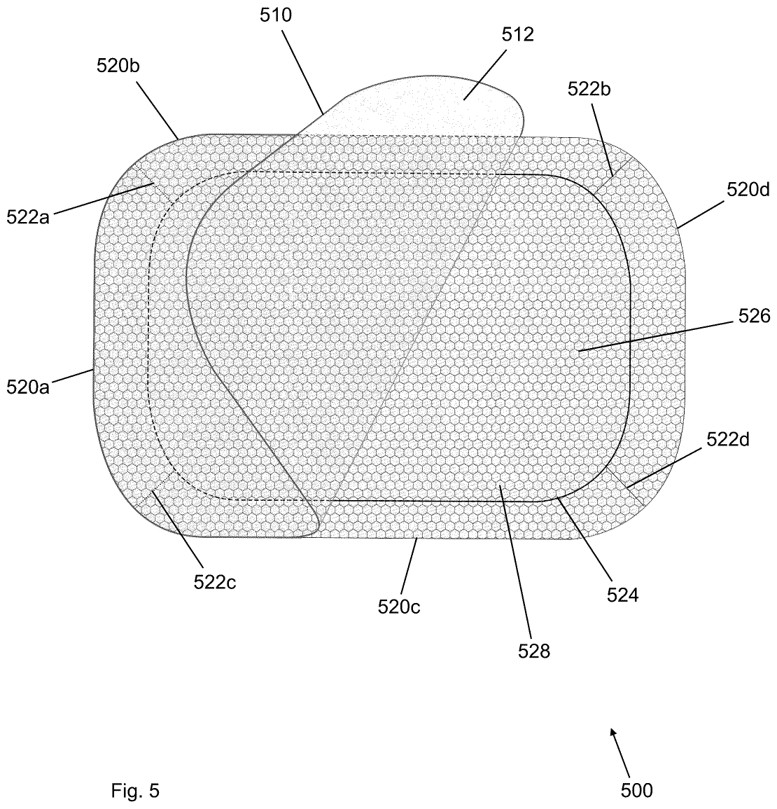

As shown in FIG. 5, an overview of a sanitary layer 510 being removed from a bottom surface of a shingles remediation belt device 500 is provided. The sanitary layer 510 may be removably coupled at one or more locations or areas across the bottom surface of the belt device 500. In some embodiments, the removable coupling may be accomplished via adhesion between the sanitary layer 510 and the bottom surface of the belt device 500 along at least a perimeter edge thereof. As illustrated in FIG. 5, the sanitary layer 510 may comprise a flexible material body 512 which allows a user to remove the sanitary layer 510 via peeling it away from the bottom surface of the belt device 500 at an edge thereof.

For example, the material body 512 may be designed with a certain degree of elasticity, providing a capacity for controlled deformation under pressure or pulling forces without breaking or tearing. Materials that can provide this elasticity might include flexible polymers or thin, flexible forms of paper, among others. This elasticity can assist in the peeling away process, as it allows the sanitary layer 510 to yield under the force of removal without tearing or leaving residues on the bottom surface of the belt device 500.

The material body 512 might also possess a level of tear resistance to prevent premature fracturing during the removal process. Furthermore, the adhesion characteristics between the sanitary layer 510 and the bottom surface of the belt device 500 should be designed to be strong enough to maintain an effective seal before use, but also allow for easy separation when the user decides to remove the sanitary layer 510. This can be achieved by using materials that have a specific surface energy, which is a property that defines the stickiness or slipperiness of a material surface. The surface energy should be low enough to ensure easy release but high enough to maintain an intact seal during storage and handling prior to use.

The bottom surface of the belt device 500 may comprise left, top, bottom and right side coupling portions 520a-520d which may be structurally segmented by associated removal elements 522a-522d and further structurally distinguished from an interior section 524 of a multilayered structure of the belt device 500 via a perforation or incision disposed around the perimeter of the interior section 524 which may serve as another removal element. Disposed across the entire bottom surface of the belt device 500 is a porous barrier layer defined by a matrix 526 and a plurality of pores 528 disposed therein which reveal an absorptive layer thereunder. The various removal elements 522a-522d may facilitate the removal of a portion of one or more layers of the multilayered structure along the perimeter of the belt device 500 such that an adhesive layer disposed therealong may become exposed after removal of the porous barrier layer, the absorptive layer, and a buffer removal layer from one or more of the coupling portions 520a-520d. This allows modular coupling of a plurality of belt devices 500 or at least a plurality of portions of belt devices 500 in order to create a composite shape and size that may accommodate remediation of a given irregularly shaped shingles rash outbreak.

As a key feature of the design, the multilayered structure in the coupling portions 520a-520d of the first belt device 500 are differentiated based on their respective roles in the adhesion process. For those coupling portions that are intended to be overlapped by a coupling portion of another belt device, the porous barrier layer, absorptive layer, and buffer removal layer are retained. This is to ensure that when these portions are overlapped, the porous barrier layer of a coupling portion of the first belt device 500 is in direct contact with the porous barrier layer of an interior section of an overlapping belt device, thereby ensuring continuity amongst separate porous barrier layers and the integrity of the therapeutic properties across the coupled belt devices. This further ensures that any coupling portion disposed over and in contact with a shingles rash outbreak retains its porous barrier layer, absorptive layer and buffer removal layer in order to remediate such the area containing the outbreak.

On the contrary, for the coupling portions that are meant to adhere to the user's skin, the porous barrier layer, the absorptive layer, and the buffer removal layer are removed. This reveals the adhesive layer, thereby enabling a secure bond with the user's skin. This approach allows for a modular coupling of multiple belt devices 500 or at least multiple portions of belt devices 500, that can be arranged to optimally conform to the irregularly shaped shingles rash outbreak. It thus facilitates the creation of a composite shape and size that can effectively remediate a given irregularly shaped shingles rash outbreak while ensuring the delivery of therapeutic properties to the affected area.

The differentiation of the layers within the coupling portions and the interior section of the belt device further ensures that the interior section, directly interacting with the shingles rash outbreak, retains all its layers. This ensures that the medicinal agents contained within the absorptive layer of the interior section are preserved and consistently delivered to the affected skin, thereby promoting efficient healing and symptom management. The unique structure and design of the coupling portions and the interior section, in this way, serves to maximize both the efficacy of the treatment and the comfort of the user.

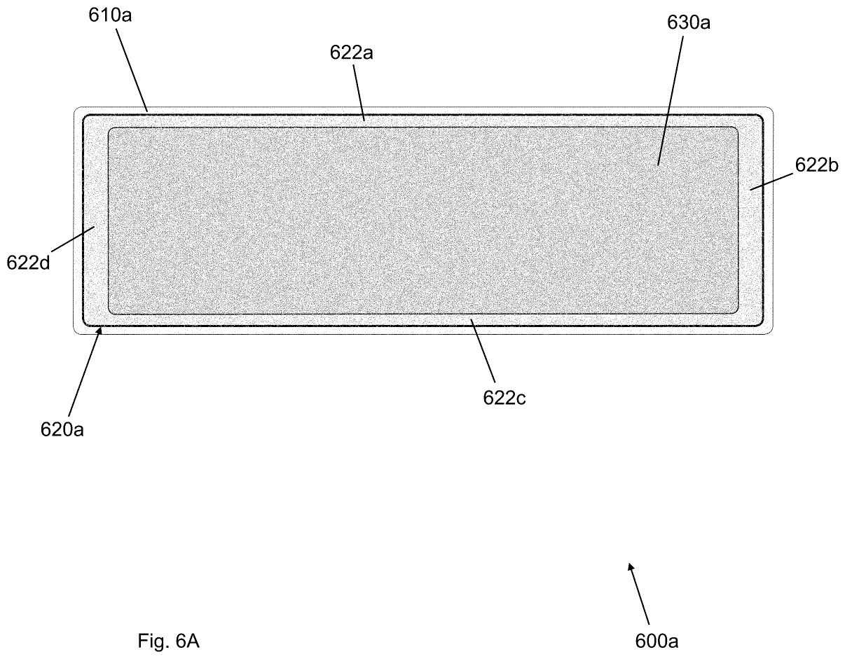

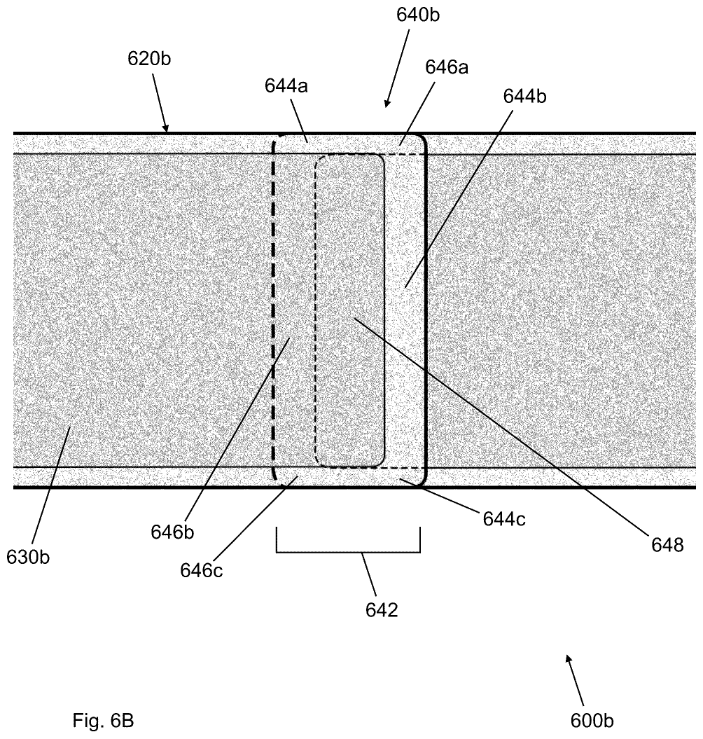

As shown in FIG. 6A, a plan view of a top surface of an elongated configuration of a shingles remediation belt device 600a is provided. The shingles remediation belt device 600a may comprise a sanitary envelope 610a enclosing and sealing a multilayer structure 620a. The multilayer structure 620a may comprise a perimeter area where an adhesive layer may be disposed such that the perimeter adhesive layer may comprise a top portion 622a, a right portion 622b, a bottom portion 622c and a left portion 622d which collectively surround an interior section 630a of the multilayered structure 620a in order to provide a mechanism for adhering the interior section 630a adjacent an area of a user's skin afflicted with a shingles rash outbreak.

The strategic design of the adhesive layer on the right and left portions 622b, 622d, having at least double the width of the top and bottom portions 622a, 622c, serves multiple purposes, paramount among which are security and comfort. The increased width in the right and left portions 622b, 622d enables a larger surface area for adhesion, creating a firm and durable connection when the belt device 600a is wrapped around the user's body. This not only increases the device's effectiveness by ensuring that it stays in place during daily movements, but it also allows for a more comfortable and adaptable fit, accommodating a range of body shapes and sizes. The larger adhesive portions thus facilitate a secure enveloping of the user's body, providing consistent contact with the affected area and supporting uninterrupted healing.

The second significant advantage lies in the cost-effectiveness achieved by minimizing the width of the top and bottom adhesive portions 622a, 622c. As these portions play a secondary role in the adhesive functionality of the device, reducing their width allows for a significant saving of adhesive material. This reduction in the consumption of resources translates into a direct cost benefit, making the production process more economical while maintaining the efficacy of the device. Additionally, this strategic distribution of the adhesive also minimizes the potential waste during the manufacturing process, contributing to an environmentally-friendly production method. Therefore, the strategic proportionality of adhesive layers not only contributes to the device's functional performance but also enhances its cost and resource efficiency.

When securing the shingles remediation belt device 600a around the user's body, it is crucial that the porous barrier layer, absorptive layer, and buffer removal layer be removed from either the left portion 622d or the right portion 622b, depending on which one is designed to overlap the other. The removal of these layers exposes the underlying adhesive layer, enabling a robust and secure adhesion between the overlapping portions. This setup ensures that the belt device 600a maintains its position around the user's body during usage, thereby enabling a continuous and effective application of treatment to the affected skin area.

Counterintuitively, it is advantageous that the portion of the device 600a that is being overlapped by the other portion may have its porous barrier layer, absorptive layer, and buffer removal layer left intact and not removed. This further distinguishes the belt device 600a from conventional wound bandages. Specifically, the intact layers on the overlapped portion offer extra remediation surface area for additional coverage of a shingles rash outbreak. In effect, this intact area continues to function as an active part of the device 600a in acting as an extension of the bottom surface of the interior section 630a of the multilayered structure 620a, thereby treating the shingles rash outbreak directly beneath it. This novel design maximizes the use of the device 600a surface area and increases its overall remediation capacity, offering users the most effective use of each belt device 600a and contributing to an efficient healing process.

The distinctive design of the shingles remediation belt device 600a also caters for instances where a shingles rash outbreak might manifest around the entire circumference of a user's body part, such as the torso or a limb. When the belt device 600a is wrapped around the user's body and the right and left overlapping portions 622b, 622d are adhered together, the portion being overlapped directly adjacent the user's skin will still retain its porous barrier layer and absorptive layer to provide a continuous 360-degree remediating coverage around the entirety of the user's body part. This comprehensive encirclement facilitates effective remediation across all areas of the outbreak, ensuring no part of the affected skin is left untreated.

Notably, the approach also mitigates potential discomfort or further irritation that might arise from requiring an adhesive end of a traditional bandage or remediation device to be disposed directly adjacent a portion of the user's skin, which itself may be afflicted with a portion of the shingles rash. The present invention overcomes this problem by allowing the overlapped portion to retain its remediating structures and effects, thereby minimizing or eliminating contact between adhesive and the shingles rash. This element of the design contributes to both the comfort and the effectiveness of the treatment process, reducing potential for additional skin irritation and providing an optimized remediation of the shingles rash.

As shown in FIG. 6B, a partial overview of two terminal ends of an elongated configuration of a shingles remediation belt device 600b secured together in an overlapping coupling configuration 640b is provided. The shingles remediation belt device 600b may comprise a multilayered structure 620b and an interior section 630b which terminate lengthwise in a first terminal end and a second terminal end. The first terminal end may overlap with the second terminal end in the overlapping coupling configuration 640b.

Specifically, the overlapping coupling configuration 640b may be defined at least in part by a first top edge 644a of the first terminal end overlapping a second top edge 646a of the second terminal end, a first side edge 644b of the first terminal end overlapping a first portion of the interior section 630b adjacent the second terminal end, a second side edge 646b of the second terminal end overlapping a second portion of the interior section 630b adjacent the first terminal end, and a first bottom edge 644c of the first terminal end overlapping a second bottom edge 646c of the second terminal end as illustrated in FIG. 6B.

The overlapping coupling configuration 640b may comprise an overlapping width region 642 and an interior overlap section 648. The degree to which the first terminal end overlaps the second terminal end, or vice versa, may be defined at least in part by the overlapping width region 642. Further, the interior overlap section 648 may be defined at least in part by the degree to which the first portion of the interior section 630b adjacent the second terminal end is overlapped by the second portion of the interior section 630b adjacent the first terminal end, or vice versa.

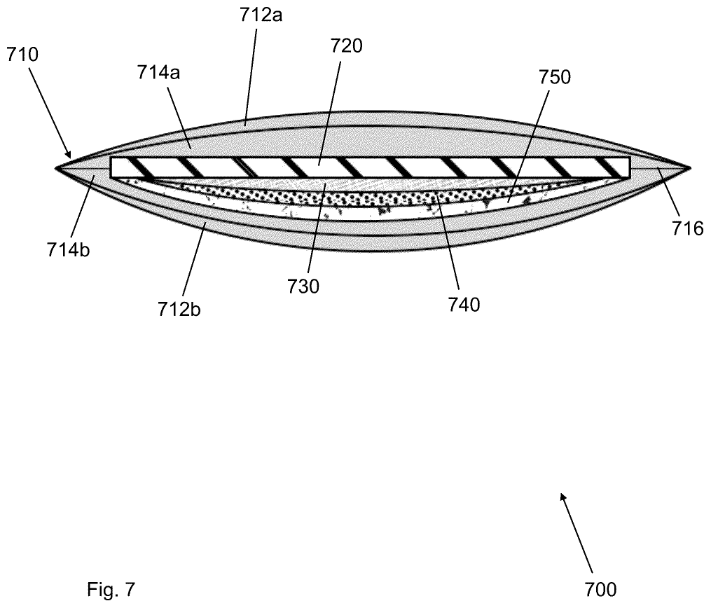

As shown in FIG. 7, a cross-sectional view of a multilayer structure of a shingles remediation belt device 700 enclosed within a product packaging envelope 710 is provided. The multilayered structure of the shingles remediation belt device 700 may comprise the product packaging envelope 710 enclosing a protective layer 720, an absorptive layer 730, a porous barrier layer 740 and a sanitary layer 750 which may be stacked in that order within the multilayered structure of the shingles remediation belt device 700 as illustrated in FIG. 7.

The product packaging envelope 710 may comprise a first outer surface 712a and a second outer surface 712b as well as a first inner surface 714a and a second inner surface 714b. Each of the first and second outer surfaces 712a, 712b and the first and second inner surfaces 714a, 714b may couple together and terminate at a perimeter seam 716 which may create a fluid tight seal around the protective layer 720, the absorptive layer 730, the porous barrier layer 740 and the sanitary layer 750 in order to maintain a sanitary environment within the product packaging envelope 710 to prevent further infection of the area of the user's skin afflicted with a shingles rash outbreak.

With regard to the specifics of the product packaging envelope 710, it is preferred that the thickness of the envelope lies within a range of about 0.1 to 1 mm. This range has been determined to be optimal for maintaining a fluid-tight seal while also allowing for easy manipulation and opening of the package. Within this range, a more preferred thickness is about 0.2 to 0.5 mm, as this offers a good balance between robustness and flexibility.

Moreover, the perimeter seam 716 of the product packaging envelope 710 should ideally exhibit a width in the range of about 2 to 10 mm. A seam of this width has been found to provide a robust and effective fluid-tight seal, while also not unnecessarily consuming packaging material. Within this range, a preferred seam width is about 3 to 5 mm, which optimizes both the integrity of the seal and the economic use of the packaging material. This measured approach to the packaging design ensures a secure and sanitary environment for the belt device 700, thereby maintaining the effectiveness of the device for shingles rash remediation.

Focusing on the protective layer 720, it is beneficial for this layer to have a thickness ranging between about 0.5 to 2 mm. A protective layer within this thickness range is capable of providing a sturdy and effective barrier against external elements, without being overly bulky or hindering the overall flexibility of the belt device 700. Within this range, a more preferred thickness of about 0.8 to 1.2 mm allows for optimal combination of durability, user comfort, and flexibility.

For the absorptive layer 730, a thickness range between about 1 to 3 mm has been found to be particularly advantageous. This range of thickness allows for adequate absorption of the medicament released from the porous barrier layer 740, and further helps in managing exudate from the shingles rash. More particularly, a preferred thickness within this range is about 1.5 to 2.5 mm which balances absorption capability with the overall bulk of the belt device 700.

Regarding the porous barrier layer 740, a thickness between about 0.5 to 1.5 mm is considered optimal. A layer in this thickness range provides a good control over the release of the medicament, while maintaining the necessary permeability for effective treatment delivery. Within this range, a more preferred thickness is about 0.7 to 1 mm, which offers an ideal balance between drug delivery control and permeability.

Finally, for the sanitary layer 750, it is beneficial for the thickness to lie in a range of about 0.1 to 1 mm. This range allows the layer to provide adequate protection for the belt device 700, without adding unnecessary bulk. Within this range, a preferred thickness of about 0.2 to 0.5 mm ensures the sanitary layer 750 remains flexible and easy for the user to remove, while still providing the required sanitary barrier.

In some embodiments of FIGS. 1-7, a shingles remediation belt device is provided comprising: a protective layer comprising an outer side surface and a inner side surface opposite the outer side surface; an absorptive layer comprising an outer side surface and a inner side surface opposite the outer side surface; a porous barrier layer comprising an outer side surface and a inner side surface opposite the outer side surface; and a sanitary layer comprising an outer side surface and an inner side surface opposite the outer side surface, wherein: the inner side surface of the protective layer is disposed adjacent the outer side surface of the absorptive layer, the inner side surface of the absorptive layer is disposed adjacent the outer side surface of the porous barrier layer, and the inner side surface of the porous barrier layer is disposed adjacent the outer side surface of the sanitary layer.

In some embodiments of FIGS. 1-7, one or more of the protective layer, the absorptive layer and the porous barrier layer comprise a plurality of coupling portions; the porous barrier layer comprises a silicone-based network structure; and the silicone-based network structure comprises one or more of a silicone elastomer mesh, a silicone-coated polypropylene mesh, a silicone-coated polyester mesh, a silicone-infused mesh dressing, a 3D printed silicone mesh, and a silicone matrix.

In some embodiments of FIGS. 1-7, the silicone-based network structure comprises a medicinal agent incorporated therein; the medicinal agent is embedded within the silicone-based network structure; the medicinal agent is impregnated within the silicone-based network structure; and the silicone-based network structure comprises a medicinal agent coated thereon.

In some embodiments of FIGS. 1-7, the porous barrier layer comprises at least one medicinal agent; the porous barrier layer comprises one or more of acrylic, nylon, polyester, hydrocolloids, petrolatum and teflon; and the absorptive layer comprises one or more of cotton, rayon, polyester, cellulose, foam, alginate fibers, hydrocolloids, hydrogel, non-woven fabric and silicone foam.

In some embodiments of FIGS. 1-7, the protective layer comprises one or more of polyethylene, polyurethane, polypropylene, polyester and thermoplastic elastomers; each of the protective layer, the absorptive layer and the porous barrier layer comprise oxygen-permeable structures; and the outer side surface of the protective layer comprises a coefficient of friction of between 0.15 and 0.4.

In some embodiments of FIGS. 1-7, the shingles remediation belt device further comprises an envelope comprising a first inner surface and a second inner surface opposite the first inner surface; the envelope is not coupled to the protective layer; the envelope is not coupled to the sanitary layer; and the envelope forms a fluid-tight seal around each of the protective layer, the absorptive layer, the porous barrier layer and the sanitary layer.

In some embodiments of FIGS. 1-7, a shingles remediation belt device is provided comprising: a protective layer comprising an outer side surface and a inner side surface opposite the outer side surface; an absorptive layer comprising an outer side surface and a inner side surface opposite the outer side surface; a porous barrier layer comprising an outer side surface and a inner side surface opposite the outer side surface; a sanitary layer comprising an outer side surface and an inner side surface opposite the outer side surface; and an envelope comprising a first inner surface and a second inner surface opposite the first inner surface, wherein: the inner side surface of the protective layer is disposed adjacent the outer side surface of the absorptive layer, the inner side surface of the absorptive layer is disposed adjacent the outer side surface of the porous barrier layer, the inner side surface of the porous barrier layer is disposed adjacent the outer side surface of the sanitary layer, the outer side surface of the protective layer is disposed adjacent the first inner surface of the envelope, and the inner side surface of the sanitary layer is disposed adjacent the second inner surface of the envelope.

In some embodiments of FIGS. 1-7, a shingles remediation belt device is provided comprising: a protective layer comprising an outer side surface and a inner side surface opposite the outer side surface; an absorptive layer comprising an outer side surface and a inner side surface opposite the outer side surface, wherein the absorptive layer is permanently coupled to the protective layer; a porous barrier layer comprising an outer side surface and a inner side surface opposite the outer side surface, wherein the porous barrier layer is permanently coupled to the protective layer; and a sanitary layer comprising an outer side surface and an inner side surface opposite the outer side surface, wherein: the sanitary layer is non-permanently coupled to the protective layer such that the sanitary layer comprises a coupled state where the sanitary layer is coupled to the protective layer and a decoupled state where the sanitary layer has been removed from the protective layer, the inner side surface of the protective layer is disposed adjacent the outer side surface of the absorptive layer, the inner side surface of the absorptive layer is disposed adjacent the outer side surface of the porous barrier layer, and the inner side surface of the porous barrier layer is disposed adjacent the outer side surface of the sanitary layer.

The specification and drawings are to be regarded in an illustrative rather than a restrictive sense. However, it will be evident that various modifications and changes may be made thereunto without departing from the broader spirit and scope of the invention as set forth in the claims. Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

All features disclosed in the specification, claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

Throughout this disclosure, the phrase ‘modularly coupled’ and similar terms and phrases are intended to convey that any element of a given class of elements may be coupled to another given element and vice versa with equal effect. For example, any extension cord of a plurality of extension cords may be modularly coupled to another extension cord and vice versa with equal effect. Further, throughout this disclosure, the phrase ‘removably coupled’ and similar terms and phrases are intended to convey that a given element may be iteratively coupled to and removed from another given element as desired. For example, a male plug of a first extension cord may be removably coupled to a female plug of a second extension cord as desired.

The use of the terms “a,” “an,” “the,” and similar referents in the context of describing the disclosed embodiments (especially in the context of the following claims) are to be construed to cover both the singular and the plural, unless otherwise indicated or clearly contradicted by context. The terms “comprising,” “having,” “including,” and “containing” are to be construed as open-ended terms (i.e., meaning “including, but not limited to,”) unless otherwise noted. The term “coupled” or “connected,” where unmodified and referring to physical connections, is to be construed as partly or wholly contained within, attached to, or joined together, even if there is something intervening. Recitation of ranges of values are merely intended to serve as a shorthand method of referring individually to each separate value falling within the range, unless otherwise indicated and each separate value is incorporated into the specification as if it were individually recited. The use of the term “set” (e.g., “a set of items”) or “subset” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal.

Conjunctive language, such as phrases of the form “at least one of A, B, and C,” or “at least one of A, B and C,” is understood with the context as used in general to present that an item, term, etc., may be either A or B or C, or any nonempty subset of the set of A and B and C, unless specifically stated otherwise or otherwise clearly contradicted by context. For instance, in the illustrative example of a set having three members, the conjunctive phrases “at least one of A, B, and C” and “at least one of A, B and C” refer to any of the following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present. In addition, unless otherwise noted or contradicted by context, the term “plurality” indicates a state of being plural (e.g., “a plurality of items” indicates multiple items). The number of items in a plurality is at least two, but can be more when so indicated either explicitly or by context.

The use of any examples, or exemplary language (e.g., “such as”) provided, is intended merely to better illuminate embodiments of the invention and does not pose a limitation on the scope of the invention unless otherwise claimed. No language in the specification should be construed as indicating any non-claimed element as essential to the practice of the invention.

Embodiments of this disclosure are described, including the best mode known to the inventors for carrying out the invention. Variations of those embodiments may become apparent to those of ordinary skill in the art upon reading the foregoing description. The inventors expect skilled artisans to employ such variations as appropriate and the inventors intend for embodiments of the present disclosure to be practiced otherwise than as specifically described. Accordingly, the scope of the present disclosure includes all modifications and equivalents of the subject matter recited in the claims appended hereto as permitted by applicable law. Moreover, although above-described elements may be described in the context of certain embodiments of the specification, unless stated otherwise or otherwise clear from context, these elements are not mutually exclusive to only those embodiments in which they are described; any combination of the above-described elements in all possible variations thereof is encompassed by the scope of the present disclosure unless otherwise indicated or otherwise clearly contradicted by context.

All references, including publications, patent applications, and patents, cited are hereby incorporated by reference to the same extent as if each reference were individually and specifically indicated to be incorporated by reference and were set forth in its entirety.

1. A shingles remediation belt device, comprising:
a protective layer comprising a outer side surface and a inner side surface opposite the outer side surface;
an absorptive layer comprising a outer side surface and a inner side surface opposite the outer side surface;
a porous barrier layer comprising a outer side surface and a inner side surface opposite the outer side surface; and
a sanitary layer comprising a outer side surface and an inner side surface opposite the outer side surface, wherein:
the inner side surface of the protective layer is disposed adjacent the outer side surface of the absorptive layer,
the inner side surface of the absorptive layer is disposed adjacent the outer side surface of the porous barrier layer, and
the inner side surface of the porous barrier layer is disposed adjacent the outer side surface of the sanitary layer.

2. The shingles remediation belt device of claim 1, wherein one or more of the protective layer, the absorptive layer and the porous barrier layer comprise a plurality of coupling portions.

3. The shingles remediation belt device of claim 1, wherein the porous barrier layer comprises a silicone-based network structure.

4. The shingles remediation belt device of claim 3, wherein the silicone-based network structure comprises one or more of a silicone elastomer mesh, a silicone-coated polypropylene mesh, a silicone-coated polyester mesh, a silicone-infused mesh dressing, a 3D printed silicone mesh, and a silicone matrix.

5. The shingles remediation belt device of claim 3, wherein the silicone-based network structure comprises a medicinal agent incorporated therein.

6. The shingles remediation belt device of claim 5, wherein the medicinal agent is embedded within the silicone-based network structure.

7. The shingles remediation belt device of claim 5, wherein the medicinal agent is impregnated within the silicone-based network structure.

8. The shingles remediation belt device of claim 3, wherein the silicone-based network structure comprises a medicinal agent coated thereon.

9. The shingles remediation belt device of claim 1, wherein the porous barrier layer comprises at least one medicinal agent.

10. The shingles remediation belt device of claim 1, wherein the porous barrier layer comprises one or more of acrylic, nylon, polyester, hydrocolloids, petrolatum and teflon.

11. The shingles remediation belt device of claim 1, wherein the absorptive layer comprises one or more of cotton, rayon, polyester, cellulose, foam, alginate fibers, hydrocolloids, hydrogel, non-woven fabric and silicone foam.

12. The shingles remediation belt device of claim 1, wherein the protective layer comprises one or more of polyethylene, polyurethane, polypropylene, polyester and thermoplastic elastomers.

13. The shingles remediation belt device of claim 1, wherein each of the protective layer, the absorptive layer and the porous barrier layer comprise oxygen-permeable structures.

14. The shingles remediation belt device of claim 1, wherein the outer side surface of the protective layer comprises a coefficient of friction of between 0.15 and 0.4.

15. The shingles remediation belt device of claim 1, further comprising: an envelope comprising a first inner surface and a second inner surface opposite the first inner surface.

16. The shingles remediation belt device of claim 15, wherein the envelope is not coupled to the protective layer.

17. The shingles remediation belt device of claim 15, wherein the envelope is not coupled to the sanitary layer.

18. The shingles remediation belt device of claim 15, wherein the envelope forms a fluid-tight seal around each of the protective layer, the absorptive layer, the porous barrier layer and the sanitary layer.

19. A shingles remediation belt device, comprising:
a protective layer comprising a outer side surface and a inner side surface opposite the outer side surface;
an absorptive layer comprising a outer side surface and a inner side surface opposite the outer side surface;
a porous barrier layer comprising a outer side surface and a inner side surface opposite the outer side surface;
a sanitary layer comprising a outer side surface and an inner side surface opposite the outer side surface; and
an envelope comprising a first inner surface and a second inner surface opposite the first inner surface, wherein:
the inner side surface of the protective layer is disposed adjacent the outer side surface of the absorptive layer,
the inner side surface of the absorptive layer is disposed adjacent the outer side surface of the porous barrier layer,
the inner side surface of the porous barrier layer is disposed adjacent the outer side surface of the sanitary layer,
the outer side surface of the protective layer is disposed adjacent the first inner surface of the envelope, and
the inner side surface of the sanitary layer is disposed adjacent the second inner surface of the envelope.

20. A shingles remediation belt device, comprising:
a protective layer comprising a outer side surface and a inner side surface opposite the outer side surface;
an absorptive layer comprising a outer side surface and a inner side surface opposite the outer side surface, wherein the absorptive layer is permanently coupled to the protective layer;
a porous barrier layer comprising a outer side surface and a inner side surface opposite the outer side surface, wherein the porous barrier layer is permanently coupled to the protective layer; and
a sanitary layer comprising a outer side surface and an inner side surface opposite the outer side surface, wherein:
the sanitary layer is non-permanently coupled to the protective layer such that the sanitary layer comprises a coupled state where the sanitary layer is coupled to the protective layer and a decoupled state where the sanitary layer has been removed from the protective layer,
the inner side surface of the protective layer is disposed adjacent the outer side surface of the absorptive layer,
the inner side surface of the absorptive layer is disposed adjacent the outer side surface of the porous barrier layer, and
the inner side surface of the porous barrier layer is disposed adjacent the outer side surface of the sanitary layer.
